(19) United States
(12) Patent Application Publication
(10) Pub. No.: US 2026/0198861 A1
(43) Pub. Date: Jul. 16, 2026

(54) DEVICES, SYSTEMS AND METHODS FOR ELECTROCARDIOGRAM DENOISING

(71) Applicant: The Administrators of The Tulane Educational Fund, New Orleans, LA (US)

(72) Inventors: Chan Ho Lim, New Orleans, LA (US); Yunsung Chung, New Orleans, LA (US); Nassir Marrouche, New Orleans, LA (US)

(21) Appl. No.: 19/451,561

(22) Filed: Jan. 16, 2026

Related U.S. Application Data
(60) Provisional application No. 63/746,119, filed on Jan. 16, 2025.

(51) Int. Cl. A61B 5/00; A61B 5/308; A61B 5/346; G16H 40/63

(52) U.S. Cl. CPC A61B 5/7203; A61B 5/308; A61B 5/346; A61B 5/7267; G16H 40/63

(57) ABSTRACT

A system may include an encoder configured receive an input signal, the encoder including a first plurality of NAF blocks. A system may include a decoder configured to generate and output signal, the decoder including a second plurality of NAF blocks. A system may include a skip connection between a first encoder NAF block of the encoder and a first decoder NAF block of the decoder, wherein the system is configured to receive noisy ECG signal data as the input signal and perform ECG signal restoration to produce the output signal.

### PRIORITY

The present application claims priority to and the benefit of U.S. Provisional Patent Application No. 63/746,119 filed Jan. 16, 2025 and titled DEVICES, SYSTEMS AND METHODS FOR ELECTROCARDIOGRAM DENOISING, the disclosure of which is incorporated herein by reference in its entirety.

### BACKGROUND

The rise of mobile electrocardiogram (ECG) devices came with the rise of frequent large magnitudes of noise in their recordings. Several artificial intelligence (AI) models have had great success in denoising, but the models' generalizability and the enhancement in clinical interpretability are still questionable.

With digital health evolution and numerous consumer electronics providing electrocardiograms (ECG), ECG denoising plays a pivotal role in standardizing and stabilizing the signals recorded amongst a multitude of devices and patients. Beyond providing a level of reliability of the mobile ECG recordings for physician's interpretation, ECG denoising can play a critical role in translating the innovative artificial intelligence approaches using 12-lead ECG signals to the digital health realm. Previously, to reach beyond traditional use cases of electrocardiograms (ECG), numerous groups across the globe have provided methods to automate the processes typically done by subject matter experts and method to augment undiscovered knowledge about ECG signal's discriminative features. For automated methods, a cardiologist-level arrhythmia detection and classification accuracy has been achieved using deep neural network. Furthermore, the clinical implications of ECG signals has been expanded by an AI model detecting low ejection fraction using 12-lead ECG signals. However, the AI models that were trained on clean 12-lead ECG in a hospital environment are bound to be inaccurate when tested with mobile ECG recorded during a patient or a consumer's daily lives. Although measuring ECG signals has become more available to the public than ever, these recordings are frequently measured without any clinical staff's oversight and more easily exposed to various types of noise. ECG recordings are prone to three main types of noise: electrode motion (EM), baseline wandering (BW) and muscle artifacts (MA). Hence, effective methods to denoise ECG signals and experiments to evaluate its enhancements in clinical interpretability and generalizability in digital health realm are imperative.

ECG denoising methods can be largely divided into two categories-traditional denoising that relies on statistical methods and deep learning-based denoising models. For example, traditional methods have seen success in ECG denoising using bandpass filters, empirical mode decomposition (EMD), Wavelet transformation methods, adaptive filtering, and Bayesian filtering methods. Recent advances in deep learning have impacted how ECG signals are processed, through new deep learning models such as autoencoders, long short-term memory (LSTM), generative adversarial network (GAN).

### SUMMARY

In some aspects, the techniques described herein relate to a denoising system, including a non-linear, activation free, feed-forward convolutional neural network (NAFNET), including: non-linear, activation free, feed-forward (NAF) blocks in a U-Net architecture including: an encoder configured receive an input signal, the encoder including a first plurality of NAF blocks, a decoder configured to generate and output signal, the decoder including a second plurality of NAF blocks, and a skip connection between a first encoder NAF block of the encoder and a first decoder NAF block of the decoder; wherein the system is configured to receive noisy ECG signal data as the input signal and perform ECG signal restoration to produce the output signal.

In some aspects, the techniques described herein relate to a method of denoising an input signal, the method including: receiving the input signal at a trained non-linear, activation free, feed-forward convolutional neural network (NAFNET) having a U-Net architecture; encoding the input signal with an encoder of the NAFNET including a first plurality of NAF blocks to create an encoded signal; decoding the encoded signal with a decoder of the NAFNET including a second plurality of NAF blocks; and outputting a denoised output signal from the decoder.

In some aspects, the techniques described herein relate to a system for ECG denoising, including a non-linear, activation free, feed-forward convolutional neural network (NAFNET), including: non-linear, activation free, feed-forward (NAF) blocks in a U-Net architecture including: an encoder configured receive an input signal, the encoder including a first plurality of NAF blocks, each NAF block of the first plurality of NAF blocks including: a mobile convolution block including: a layer normalization layer, a pointwise convolution layer, a depth wise convolution layer, a simple channel attention layer, a simple gate, an elementwise multiplication/addition layer, and a dropout layer, and a feed-forward network block including: a layer normalization layer, a pointwise convolution layer, a depth wise convolution layer, a simple gate, and a dropout layer; and a decoder configured to generate and output signal, the decoder including a second plurality of NAF blocks; and a skip connection between a first encoder NAF block of the encoder and a first decoder NAF block of the decoder; wherein the system is configured to receive noisy ECG signal data as the input signal and perform ECG signal restoration to produce the output signal.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and aspects of embodiments of the disclosure will be set forth in the description which follows, and in part will be obvious from the description, or may be learned by the practice of such embodiments. The features and aspects of such embodiments may be realized and obtained by means of the instruments and combinations particularly pointed out in the appended claims. These and other features will become more fully apparent from the following description and appended claims or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, non-schematic drawings should be considered as being to scale for some embodiments of the present disclosure, but not to scale for other embodiments contemplated herein. Understanding that the drawings depict some example embodiments, the embodiments will be described and explained with additional specificity and detail through the use of the accompanying drawings in which:

FIG. 1 is a diagram of an architecture of the NAFNET configured to denoise an input signal, according to at least some embodiments of the present disclosure.

FIG. 2 is a schematic illustration of layers of a NAF block, according to at least some embodiments of the present disclosure.

FIG. 3 is a chart illustrating experimental data of denoised signals compared to original clean signals and noisy signals containing simulated noise for training and validation purposes, according to at least some embodiments of the present disclosure.

FIG. 4 is a flowchart illustrating a method of denoising an input signal, according to at least some embodiments of the present disclosure.

FIG. 5 is a chart illustrating four examples of experimental data of denoised signals from single lead input signals.

### DETAILED DESCRIPTION

Embodiments of the present disclosure generally relate to denoising an input signal to produce a denoised output signal. More particularly, the methods and systems described herein related to denoising an electrocardiogram (ECG) signal to produce an output ECG with a greater signal-to-noise ratio (SNR) than the input ECG signal. In some embodiments, the systems and methods according to the present disclosure denoise single-lead ECG signals, such as those collected by or from consumer wearable electronics, and produces a denoised ECG signal for clinical use.

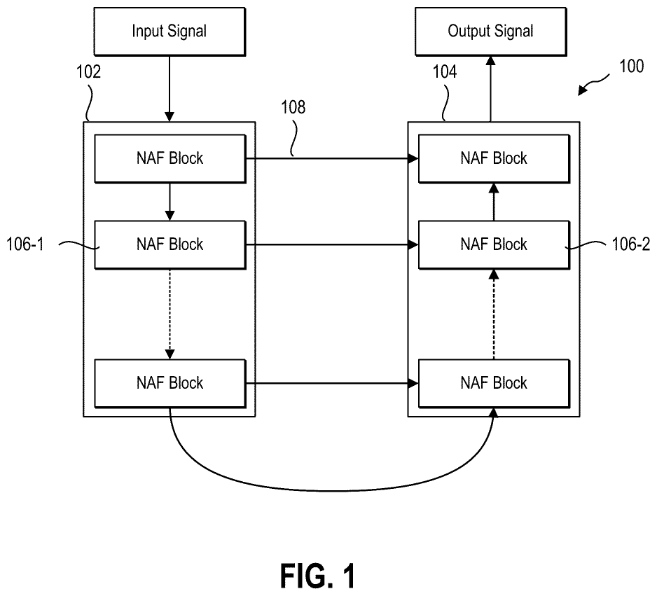

In some embodiments, the systems and methods for denoising an input signal include a non-linear, activation free, feed-forward convolutional neural network (NAFNET) that includes an encoder that alters a resolution of the input signal and a decoder that restores the resolution to at least part of the original resolution of the input signal. FIG. 1 is a diagram illustrating an embodiment of an architecture of the NAFNET 100 configured to denoise an input signal.

In some embodiments, the NAFNET 100 has a U-Net architecture in which an encoder 102 and a decoder 104 are connected to pass encoded signals therebetween. In some embodiments, the U-Net architecture connects the encoder non-linear, activation free, feed-forward (NAF) blocks 106-1 of a first plurality of NAF blocks of the encoder 102 to the decoder NAF blocks 106-2 of a second plurality of NAF blocks of the decoder 104 sequentially. In some embodiments, the U-Net architecture of the NAFNET connects a first encoder NAF block 106-1 to a first decoder NAF block 106-2 via a skip connection 108. In some embodiments, the U-Net architecture connects the encoder NAF blocks 106-1 to the decoder NAF blocks 106-2 both sequentially and via skip connections 108. For example, the NAFNET 100 may include a plurality of skip connections 108 connecting encoder NAF blocks 106-1 to decoder NAF blocks 106-2. The NAFNET 100 may, therefore, pass encoded signals at different resolutions from the encoder 102 at different encoder NAF blocks 106-1 to the decoder NAF blocks 106-2 of the decoder 104, allowing denoising of different degrees using the NAFNET 100.

A NAFNet according to the present disclosure has reduced complexity and dimension relative to conventional NAFNET architecture to transform the conventional framework dedicated to 2-dimensional image restoration to ECG signal restoration. In some embodiments, the encoder 102 includes at least 10 encoder NAF Blocks 106-1. In some embodiments, the decoder 104 includes at least 4 decoder NAF Blocks 106-2. In some embodiments, the encoder 102 includes at least 10 encoder NAF Blocks 106-1 and the decoder 104 includes at least 4 decoder NAF Blocks 106-2.

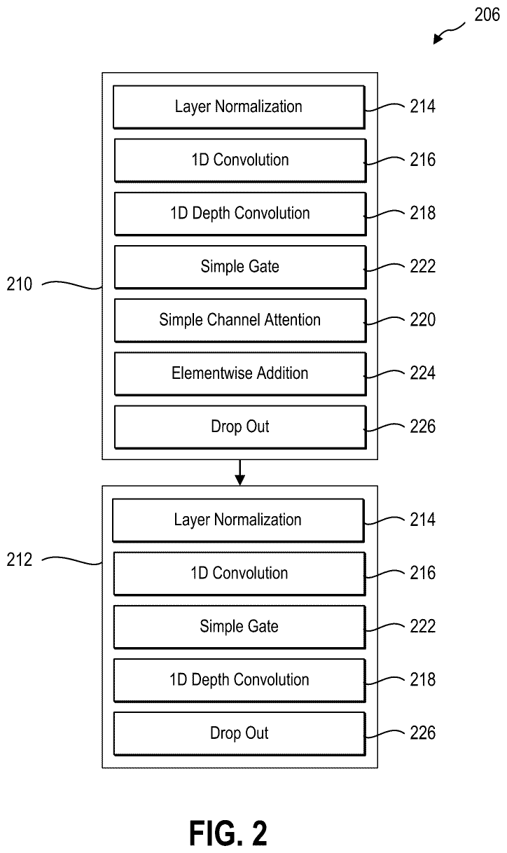

FIG. 2 is a schematic illustration of an embodiment of layers of a NAF block 206. In some embodiments, the NAF block 206 includes layers without nonlinear activation functions (e.g., sigmoid, softmax, ReLu, etc.). The NAF block 206 includes a mobile convolution (MB) block 210 and a feed-forward network (FFN) block 212. In some embodiments, the MB block 210 includes a layer normalization layer 214, a pointwise (1D) convolution layer 216, a (1D) depth wise convolution layer 218, a simple channel attention (SCA) layer 220, a simple gate layer 222, an elementwise multiplication/addition layer 224, and a dropout layer 226. In some embodiments, the layers are in the order described above. In some embodiments, the MB block 210 includes one or more additional layers located in the sequence of layers described above. In some embodiments, the MB block 210 includes layers in a different order than described above.

The FFN block 212 includes at least some of the same layers as the MB block 210. For example, the FFN block 212 may include a layer normalization layer 214, a pointwise (1D) convolution layer 216, a simple gate layer 222, an elementwise multiplication/addition layer 224, and a dropout layer 226. In some embodiments, the layers are in the order described above. In some embodiments, the FFN block 212 includes one or more additional layers located in the sequence of layers described above. In some embodiments, the FFN block 212 includes layers in a different order than described above.

An example difference between an embodiment of a NAF Block 206 according to the present disclosure versus the feed forward networks in conventional transformers is in the simple gate layer 222, which allows the entire NAF Block 206 to be free of nonlinear activation functions.

In some embodiments, a NAFNET (such as that described in relation to FIG. 1) including the NAF blocks (such as those described in relation to FIG. 2) is a trained NAFNET. In some embodiments, training the NAFNet includes receiving batches that comprise pairs of noisy ECG signal generated from the preprocessing steps and their corresponding original ECG strips unaltered by noise. The matching original ECG signals are only used to calculate the loss by taking the distance of the denoised output to the original signal.

A Mean Squared Error (MSE) L_{MSE} is adopted to measure the differences between denoised signals and clean signals. Lₘₐₓ is used to measure the maximum difference between denoised and clean signals. The Lₘₐₓ value allows the NAFNET to capture the local characteristics of ECG signals.

$${L}_{MSE} = \frac{1}{N} {\sum }_{n = 1}^{N} ( {\tilde{x}}_{n} - {x}_{n} )$$

$${L}_{m ax} = max ( \vert {\hat{x}}_{1} - {x}_{1} \vert , \vert {\hat{x}}_{2} - {x}_{2} \vert , ⋯ \vert {\hat{x}}_{N} - {x}_{N} \vert )$$

where \hat{x} indicates denoised signals and x indicates clean signals. N represents the total number of samples. The total loss function is defined as:

$$L = {λ}_{1} {L}_{MSE} + {λ}_{2} {L}_{m ax}$$

where λ₁ and λ₂ are weighted coefficients. Through experimental testing, λ₁=0.8 and λ₂=0.2 were chosen for stability and reliability. In some embodiments, the NAFNET is trained with an optimizer with learning rate of 0.0001 (β₁=0.9, β₂=0.999). In some embodiments, the NAFNET is trained with a batch size of 256.

In some embodiments, training a NAFNET according to present disclosure includes generation of training data from known clean ECG signals. In some embodiments, combinations of noise are introduced into the clean ECG samples from an arrhythmia database. The added noise allows a training dataset with three related variations of each ECG signal—the clean ECG samples, the injected noise, and the simulated noisy ECG sample. In some embodiments, a NAFNet according to the present disclosure receives simulated noisy ECG samples and generate denoised samples that resemble the corresponding original clean ECG samples as closely as possible. When introducing noise into the original ECG signals, the signal to noise ratio (SNR) is measured by the following equation:

$$SNR = 10 {log}_{10} [ \frac{{\sum }_{i = 0}^{N} {x}_{i}^{2}}{{\sum }_{i = 0}^{N} {n}_{i}^{2}} ]$$

The equation above generates various mixtures of noise by providing a random length of a random signal to noise ratio (SNR) to a randomized segment in a selected ECG signal. For validating samples, for examples, 42 different testing environments were created by fixing the signal to noise to integers from 0 dB to 5 dB and providing all combinations of baseline wander (BW), muscle artifacts (MA), and electrode motion (EM) artifacts and/or noise. In some embodiments, the signal to noise of generated noisy signals is fixed by calculating a that is provided by the following equation:

$$\dot{x} = {x}_{i} + {n}_{i} * α and :$$

$$α = \sqrt{\frac{{\sum }_{i = 0}^{N} {x}_{i}^{2}}{{\sum }_{i = 0}^{N} {n}_{i} * 1 {0}^{( SNR / 10 )}}}$$

where \dot{x} represents the simulated noisy signals, x represents individual clean ECG sample from the arrhythmia database, n is the noise, N is the number of samples, and a represents the constant that is multiplied to the noise to generate noisy samples at a fixed SNR.

With the formulas above, combinations of simulated noisy ECG samples at fixed SNR from 0 dB to 5 dB with all combinations of noise types are generated. The signals were then normalized using min-max normalization:

$$Norm ( x ) = \frac{{x}_{n} - {x}_{m i n}}{{x}_{ma x} - {x}_{m i n}}$$

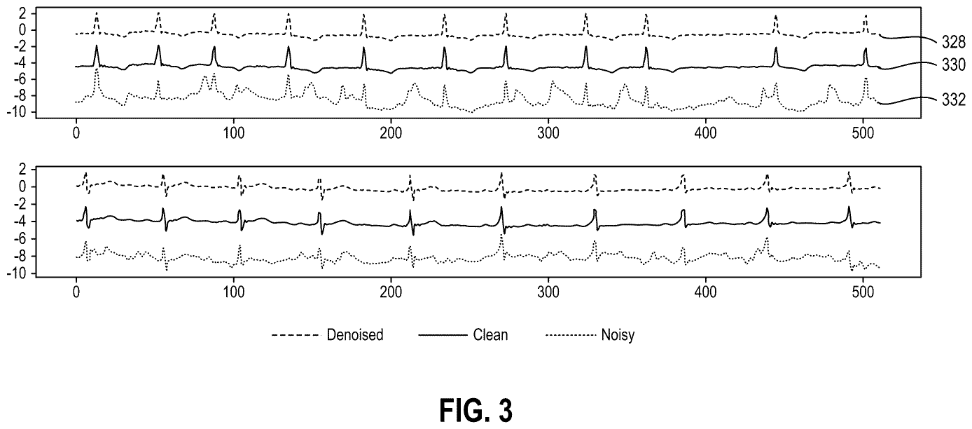

FIG. 3 is a chart illustrating an example of experimental data of denoised signals 328 compared to original clean signals 330 and noisy signals 332 containing simulated noise for training and validation purposes. As can be seen in the chart, the noisy signals 332 introduce BW, MA, and EM noise to the clean signals 330, and the NAFNET reconstructs a denoised signal 328 that is a close approximation of the original clean signal 330.

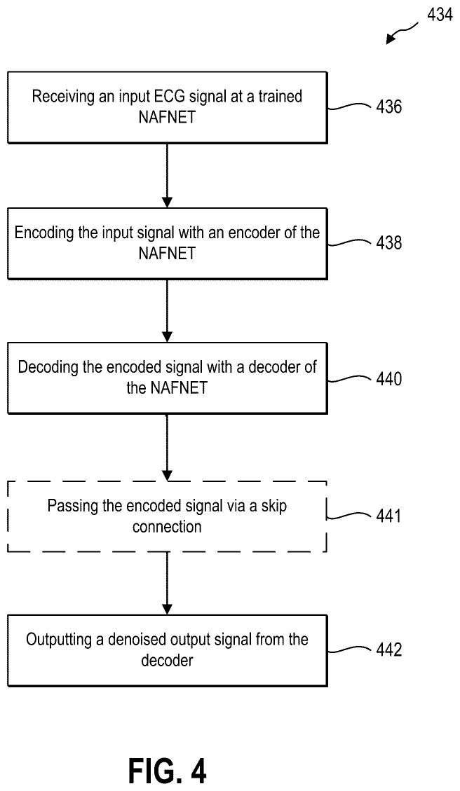

FIG. 4 is a flowchart illustrating a method 434 of denoising an input signal. The method 434 includes receiving the input signal at a trained NAFNET having a U-Net architecture at 436.

In some embodiments, the NAFNET is according to any NAFNET described herein. For example, the NAFNET may include an encoder and/or decoder as described in relation to FIG. 1. In some examples, an encoder and/or decoder of the NAFNET includes any or all of the layers described in relation to FIG. 2. For example, the encoder and/or decoder may include a plurality of NAF blocks containing an MB block and/or an FFN block. In some embodiments, the MB block includes a layer normalization layer, a pointwise convolution layer, a depth wise convolution layer, a simple channel attention (SCA) layer, a simple gate layer, an elementwise multiplication/addition layer, a dropout layer, or combinations thereof. In some embodiments, the FFN block includes at least some of the same layers as the MB block. For example, the FFN block may include a layer normalization layer, a pointwise convolution layer, a simple gate layer, an elementwise multiplication/addition layer, and a dropout layer.

In some examples, the input signal has a frequency of 200 Hz. In some examples, the input signal has a frequency of no more than 200 Hz. In some examples, the input signal has a length of 20 seconds. In some examples, the input signal has a length of no more than 20 seconds. In some embodiments, the input signal is an ECG signal that includes one or more of EM, BW, and MA noise.

In some embodiments, the method 434 further includes encoding the input signal with an encoder of the NAFNET including a first plurality of NAF blocks to create an encoded signal at 438. In some examples, encoding the input signal includes altering a resolution of the input signal to create the encoded signal. In some examples, encoding the input signal includes reducing a resolution of the input signal to create the encoded signal. In some examples, encoding the input signal includes increasing a resolution of the input signal to create the encoded signal.

In some embodiments, the method 434 further includes decoding the encoded signal with a decoder of the NAFNET including a second plurality of NAF blocks at 440. In some examples, decoding the encoded signal includes altering a resolution of the encoded signal to create a denoised signal. In some examples, decoding the encoded signal includes reducing a resolution of the encoded signal to create the denoised signal. In some examples, decoding the encoded signal includes increasing a resolution of the encoded signal to create the denoised signal.

In some examples, the encoded signal is produced by a plurality of encoder NAF blocks. In some examples, the encoded signal is produced by encoding the signal sequentially through all of the encoder NAF blocks of the encoder. In some embodiments, the method includes passing the encoded signal to the decoder through a skip connection after less than all of the encoder NAF blocks at 441.

In some embodiments, the method 434 further includes outputting a denoised output signal from the decoder at 442. In some embodiments, outputting the denoised output signal includes storing the denoised output signal to a database. In some embodiments, outputting the denoised output signal includes transmitting the denoised output signal to a patient. In some embodiments, outputting the denoised output signal includes transmitting the denoised output signal to a clinician or doctor. In some embodiments, the denoised output signal has a different resolution than the input signal. In some embodiments, the denoised output signal has a lower resolution than the input signal.

In some embodiments, the method optionally includes measuring a performance of the denoising by root mean square error (RMSE) and SNR as follows:

$$RMSE = \sqrt{\frac{1}{N} {\sum }_{n = 1}^{N} ( {\tilde{x}}_{n} - {x}_{n} )}$$

$$SNR = 10 {log}_{10} \frac{{\sum }_{n = 1}^{N} {x}_{n}^{2}}{{( {\tilde{x}}_{n} - {x}_{n} )}^{2}}$$

where x is the original clean signal, \tilde{x} is denoised signal, and N is the number of samples. RMSE indicates the difference between two signals.

While the SNR formula for the evaluation is different from the SNR equation described in relation to preparation of the training samples, both formulas essentially represent the same ratio of the ECG signal to the noise as \tilde{x}-x is the remaining noise after the signal is denoised. The RMSE and SNR possess an inverse relationship where smaller RMSE values indicate larger SNR. In some embodiments, the objective of a NAFNet according to the present disclosure is to minimize RMSE and maximize SNR, which indicates an improved ECG signal to the noise in the signal. In some embodiments, the RMSE and/or SNR is measured at a plurality of times in the method of denoising described in relation to FIG. 4. In some embodiment, the RMSE and/or SNR is used to determine when to pass an encoded signal via a skip connection. For example, when the RMSE and/or SNR exceeds a threshold value, the system may pass the encoded signal to the decoder.

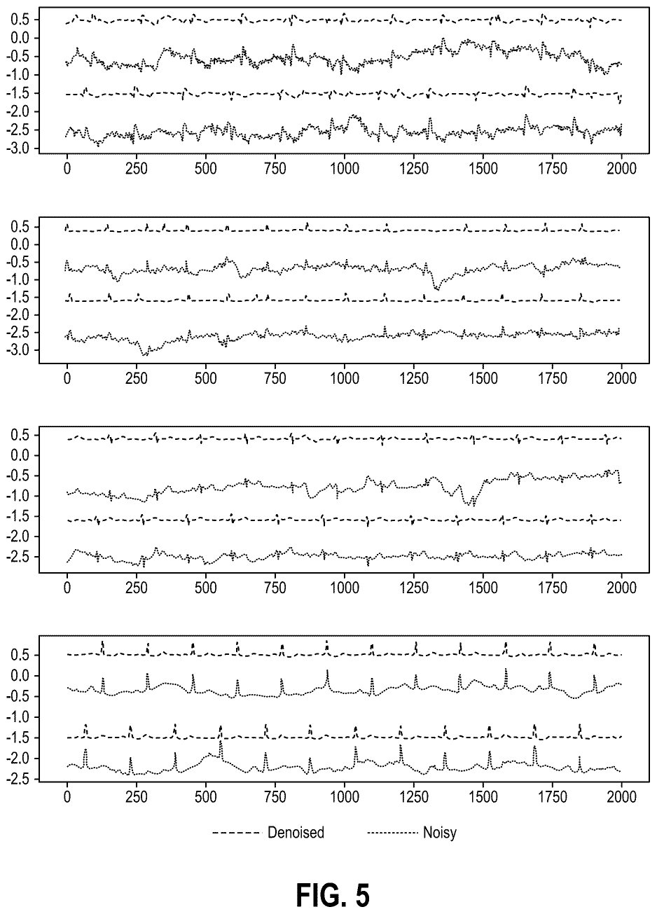

FIG. 5 is a chart illustrating four examples of experimental data of denoised signals from single lead input signals. The single lead input signals were retrieved from an independent dataset from a publicly available Delayed Enhancement MRI and Atrial Fibrillation Catheter Ablation (DECAAF-II) study. An embodiment of a NAFNet according to the present disclosure was trained as described herein based on samples from DECAAF-II, which are measured at 200 Hz with a 20 second window, which provides a sample length of 4000. The training data was generated using the same framework as the second experiment, but with sampling rate of 200 Hz and sample length of 20 seconds.

The denoised outputs from Cardio-NAFNet are in green and the unfiltered mobile ECG signals from DECAAF-II trial are in red. The records are in an order by their original signal quality score ranging from one to four. The records illustrate a strong improvement in clarity of the ECG signals after denoising, allowing reliable interpretation of the single lead ECG signals.

### INDUSTRIAL APPLICABILITY

The following description includes various embodiments that, where feasible, may be combined in any permutation. For example, the embodiment of the immediately following paragraph may be combined with any or all embodiments of the following paragraphs. Embodiments that describe acts of a method may be combined with embodiments that describe, for example, systems and/or devices. Any permutation of the following paragraphs is considered to be hereby disclosed for the purposes of providing “unambiguously derivable support” for any claim amendment based on the following paragraphs. Furthermore, the following paragraphs provide support such that any combination of the following paragraphs would not create an “intermediate generalization.”

Clause 1. A denoising system, comprising a non-linear, activation free, feed-forward convolutional neural network (NAFNET), comprising: non-linear, activation free, feed-forward (NAF) blocks in a U-Net architecture comprising: an encoder configured receive an input signal, the encoder including a first plurality of NAF blocks, a decoder configured to generate and output signal, the decoder including a second plurality of NAF blocks, and a skip connection between a first encoder NAF block of the encoder and a first decoder NAF block of the decoder; wherein the system is configured to receive noisy ECG signal data as the input signal and perform ECG signal restoration to produce the output signal.

Clause 2. The denoising system of clause 1, wherein each NAF block of the encoder includes a mobile convolution block (MB block) and a feed forward network (FFN) block.

Clause 3. The denoising system of clause 2, wherein the MB block includes: a layer normalization layer, a pointwise convolution layer, a depth wise convolution layer, a simple channel attention layer, a simple gate, an elementwise multiplication/addition layer, and a dropout layer.

Clause 4. The denoising system of clause 2, wherein the FFN block includes: a layer normalization layer, a pointwise convolution layer, a depth wise convolution layer, a simple gate, and a dropout layer.

Clause 5. The denoising system of any preceding clause, wherein the first plurality of NAF blocks includes at least 10 NAF blocks.

Clause 6. The denoising system of any preceding clause, wherein the second plurality of NAF blocks includes at least 4 NAF blocks.

Clause 7. The denoising system of any preceding clause, wherein the first plurality of NAF blocks includes the same number of NAF blocks as the second plurality of NAF blocks.

Clause 8. The denoising system of clause 1 further comprising a mean square error loss function block configured to measure a difference between the output signal and a clean signal.

Clause 9. The denoising system of clause 8, wherein the loss function is according to:

Clause 10. The denoising system of clause 9, wherein is 0.8 and is 0.2.

Clause 11. The denoising system of any preceding clause, wherein the system is trained with a learning rate of 0.0001.

Clause 12. The denoising system of any preceding clause, wherein the input signal has a frequency no more than 200 Hz.

Clause 13. The denoising system of any preceding clause, wherein the input signal has a length no more than 20 seconds.

Clause 14. The denoising system of any preceding clause, wherein the skip connection is a first skip connection, and further comprising a second skip connection between a second encoder NAF block of the encoder and a second decoder NAF block of the decoder.

Clause 15. The denoising system of any preceding clause, wherein the input signal includes electrode motion (EM), baseline wandering (BW) and muscle artifacts (MA) noise.

Clause 16. A method of denoising an input signal, the method comprising: receiving the input signal at a trained non-linear, activation free, feed-forward convolutional neural network (NAFNET) having a U-Net architecture; encoding the input signal with an encoder of the NAFNET including a first plurality of NAF blocks to create an encoded signal; decoding the encoded signal with a decoder of the NAFNET including a second plurality of NAF blocks; and outputting a denoised output signal from the decoder.

Clause 17. The method of clause 16, wherein the input signal is a single-lead ECG signal.

Clause 18. The method of clause 16 or 17, wherein the input signal has a 200 Hz frequency.

Clause 19. The method of any of clauses 16-18, further comprising preprocessing the input signal by sampling the input signal at a 200 Hz frequency.

Clause 20. A system for ECG denoising, comprising a non-linear, activation free, feed-forward convolutional neural network (NAFNET), comprising: non-linear, activation free, feed-forward (NAF) blocks in a U-Net architecture comprising: an encoder configured receive an input signal, the encoder including a first plurality of NAF blocks, each NAF block of the first plurality of NAF blocks including: a mobile convolution block including: a layer normalization layer, a pointwise convolution layer, a depth wise convolution layer, a simple channel attention layer, a simple gate, an elementwise multiplication/addition layer, and a dropout layer, and a feed-forward network block including: a layer normalization layer, a pointwise convolution layer, a depth wise convolution layer, a simple gate, and a dropout layer; and a decoder configured to generate and output signal, the decoder including a second plurality of NAF blocks; and a skip connection between a first encoder NAF block of the encoder and a first decoder NAF block of the decoder; wherein the system is configured to receive noisy ECG signal data as the input signal and perform ECG signal restoration to produce the output signal.

It should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein, to the extent such features are not described as being mutually exclusive. Numbers, percentages, ratios, or other values stated herein are intended to include that value, and also other values that are “about”, “substantially”, or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims. The described embodiments are therefore to be considered as illustrative and not restrictive, and the scope of the disclosure is indicated by the appended claims rather than by the foregoing description.

What is claimed is:

1. A denoising system, comprising a non-linear, activation free, feed-forward convolutional neural network (NAFNET), comprising:
non-linear, activation free, feed-forward (NAF) blocks in a U-Net architecture comprising:
an encoder configured receive an input signal, the encoder including a first plurality of NAF blocks,
a decoder configured to generate and output signal, the decoder including a second plurality of NAF blocks, and
a skip connection between a first encoder NAF block of the encoder and a first decoder NAF block of the decoder;
wherein the system is configured to receive noisy ECG signal data as the input signal and perform ECG signal restoration to produce the output signal.

2. The denoising system of claim 1, wherein each NAF block of the encoder includes a mobile convolution block (MB block) and a feed forward network (FFN) block.

3. The denoising system of claim 2, wherein the MB block includes:
a layer normalization layer,
a pointwise convolution layer,
a depth wise convolution layer,
a simple channel attention layer,
a simple gate,
an elementwise multiplication/addition layer, and
a dropout layer.

4. The denoising system of claim 2, wherein the FFN block includes:
a layer normalization layer,
a pointwise convolution layer,
a depth wise convolution layer,
a simple gate, and
a dropout layer.

5. The denoising system of claim 1, wherein the first plurality of NAF blocks includes at least 10 NAF blocks.

6. The denoising system of claim 1, wherein the second plurality of NAF blocks includes at least 4 NAF blocks.

7. The denoising system of claim 1, wherein the first plurality of NAF blocks includes the same number of NAF blocks as the second plurality of NAF blocks.

8. The denoising system of claim 1 further comprising a mean square error loss function block configured to measure a difference between the output signal and a clean signal.

9. The denoising system of claim 8, wherein the loss function is according to:
$$L = {λ}_{1} {L}_{MSE} + {λ}_{2} {L}_{ma x}$$

10. The denoising system of claim 9, wherein λ₁ is 0.8 and λ₂ is 0.2.

11. The denoising system of claim 1, wherein the system is trained with a learning rate of 0.0001.

12. The denoising system of claim 1, wherein the input signal has a frequency no more than 200 Hz.

13. The denoising system of claim 1, wherein the input signal has a length no more than 20 seconds.

14. The denoising system of claim 1, wherein the skip connection is a first skip connection, and further comprising a second skip connection between a second encoder NAF block of the encoder and a second decoder NAF block of the decoder.

15. The denoising system of claim 1, wherein the input signal includes electrode motion (EM), baseline wandering (BW) and muscle artifacts (MA) noise.

16. A method of denoising an input signal, the method comprising:
receiving the input signal at a trained non-linear, activation free, feed-forward convolutional neural network (NAFNET) having a U-Net architecture;
encoding the input signal with an encoder of the NAFNET including a first plurality of NAF blocks to create an encoded signal;
decoding the encoded signal with a decoder of the NAFNET including a second plurality of NAF blocks; and
outputting a denoised output signal from the decoder.

17. The method of claim 16, wherein the input signal is a single-lead ECG signal.

18. The method of claim 16, wherein the input signal has a 200 Hz frequency.

19. The method of claim 16, further comprising preprocessing the input signal by sampling the input signal at a 200 Hz frequency.

20. A system for ECG denoising, comprising a non-linear, activation free, feed-forward convolutional neural network (NAFNET), comprising:
non-linear, activation free, feed-forward (NAF) blocks in a U-Net architecture comprising:
an encoder configured receive an input signal, the encoder including a first plurality of NAF blocks, each NAF block of the first plurality of NAF blocks including:
a mobile convolution block including:
a layer normalization layer,
a pointwise convolution layer,
a depth wise convolution layer,
a simple channel attention layer,
a simple gate,
an elementwise multiplication/addition layer, and
a dropout layer, and
a feed-forward network block including:
a layer normalization layer,
a pointwise convolution layer,
a depth wise convolution layer,
a simple gate, and
a dropout layer; and
a decoder configured to generate and output signal, the decoder including a second plurality of NAF blocks; and
a skip connection between a first encoder NAF block of the encoder and a first decoder NAF block of the decoder;
wherein the system is configured to receive noisy ECG signal data as the input signal and perform ECG signal restoration to produce the output signal.
